(19) United States
(12) Patent Application Publication
(10) Pub. No.: US 2010/0187131 A1
(43) Pub. Date: Jul. 29, 2010

(54) PLATFORM TECHNOLOGY FOR DETECTING MICROORGANISMS

(75) Inventors: Peter Faguy, Rochester Hills, MI (US); David M. Faguy, Albuquerque, NM (US)

Correspondence Address: BROOKS KUSHMAN P.C., 1000 TOWN CENTER, TWENTY-SECOND FLOOR, SOUTHFIELD, MI, 48075, US

(73) Assignee: DELTA BIOSENSING, LLC, Rochester Hills, MI (US)

(21) Appl. No.: 12/667,940

(22) PCT Filed: Jul. 7, 2008
(86) PCT No.: PCT/US08/69346

§ 371 Date: Jan. 6, 2010

Related U.S. Application Data
(60) Provisional application No. 60/948,440, filed on Jul. 6, 2007.

(51) Int. Cl. G01N 33/50

(52) U.S. Cl. 205/777.5; 204/403.01

(57) ABSTRACT

An apparatus for detecting the presence of a microorganism in a microbial sample includes a first enclosable chamber for holding a first portion of a microbial sample. The first enclosable chamber holds the first portion of the microbial sample in a manner that allows a gaseous region to be formed therein thereby defining an interface between the gaseous region and the first portion. The apparatus of this embodiment further includes a first metabolic compound monitor in communication with the gaseous region. The first metabolic compound monitor provides a signal functionally dependent on metabolic compound concentration in the gaseous region wherein the signal allows identification of a metabolic compound rich state and metabolic compound depleted state such that at some point during a predetermined period of time a transition between the metabolic compound rich state and the metabolic compound depleted state occurs. The method executed by the apparatus is also provided.

### CROSS-REFERENCE TO RELATED APPLICATIONS

This application claims the benefit of U.S. provisional application Ser. No. 60/948,440 filed Jul. 6, 2007. The entire disclosure of this application is hereby incorporated by reference.

### BACKGROUND OF THE INVENTION

1. Field of the Invention

The present invention is related to methods and an apparatus for detecting the presence of a microbe in a sample.

2. Background Art

For a number of practical applications, such as clinical diagnosis, bioterrorist threat assessment, food safety testing and environmental monitoring, it is desirable to detect the presence and quantity of a specific microorganism(s). A tested specimen frequently contains a large number of microorganisms from which a specific target microorganism or a group of specifically targeted microorganisms must be specifically and rapidly detected. Examples of microorganism-specific tests are found, for example, in U.S. Pat. Nos. 5,498,524 to Rees, et al., 6,436,661 to Adams, et al., and 6,809,180 to de Boer, et al.

U.S. Patent Publication No.: US 2004/0175780A1 by Li et al. describes a method for quantifying respiring microorganisms through their consumption of oxygen. Oxygen concentration is determined amperometrically. The tests described in this patent application are large volume, e.g., 15 ml. per test, time-consuming, e.g., upwards of two hours. The tests described by Li et al. would not be particularly organism-specific if multiple microorganisms are present, as is generally the case.

U.S. Pat. No. 6,461,833 to Wilson describes a method for assessing the presence of a particular bacterium in a sample also containing a second bacterium. This is done using bacteriophage infection of the first bacterium and subsequent manipulation of the reproduced bacteriophage detectable through plaque formation. The tests described by Wilson require overnight incubation and have multiple sample/biomaterials manipulation steps that must be done in a microbiology laboratory environment.

Accordingly, there exists a need for methods and apparatuses that provide accurate, rapid, specific, inexpensive and reproducible detection of microorganisms.

### SUMMARY OF THE INVENTION

The present invention solves one or more problems of the prior art by providing in at least one embodiment a method and apparatus for detecting the presence of a microorganism in a microbial sample. The microbial sample includes the microorganism and a growth medium while the detected microorganism characteristically produces or consumes a metabolic compound. The apparatus of this embodiment includes a first enclosable chamber for holding a first portion of the microbial sample. The first enclosable chamber holds the first portion of the microbial sample in a manner that allows a first gaseous region to be formed therein thereby defining an interface between the first gaseous region and the first portion. The apparatus of this embodiment further includes a first metabolic compound monitor in communication with the gaseous region. The first metabolic compound monitor provides a signal functionally dependent on metabolic compound concentration in the first gaseous region wherein the signal allows identification of a metabolic compound rich state and metabolic compound depleted state such that at some point during a predetermined period of time a transition between the metabolic compound rich state and the metabolic compound depleted state occurs.

In another embodiment, a method of forming microbe-detecting devices is provided. The method of this embodiment comprises joining an electrode containing section with a growth medium container section via a semipermeable membrane to form a microbe-detecting apparatus.

### BRIEF DESCRIPTION OF THE DRAWINGS

FIG. 1A is a schematic illustration of an embodiment of a microbe-detecting apparatus;

FIG. 1B is a schematic illustration of another embodiment of a microbe-detecting apparatus;

FIG. 2A is an idealized plot of the signal generated from a variation of the microbe-detecting apparatus;

FIG. 2B is an idealized plot of the signal generated from a variation of the microbe-detecting apparatus;

FIG. 3 is a schematic illustration of an embodiment of a microbe-detecting apparatus having a plurality of enclosable chambers;

FIG. 4A is a schematic cross-section of an embodiment of an apparatus for detecting the presence of a microorganism in a microbial sample utilizing an electrochemical cell;

FIG. 4B is a schematic cross-section of another embodiment of an apparatus for detecting the presence of a microorganism in a microbial sample utilizing an electrochemical cell;

FIG. 5A is a top view of a substrate with electrodes disposed thereon that is useful in variation of a microbe-detecting apparatus;

FIG. 5B is a top view of a substrate with an enclosable chamber that is useful in a variation of a microbe-detecting apparatus;

FIG. 6 provides a series of cross-sectional views illustrating an embodiment for forming an enclosable microbe-detecting apparatus set forth; and

FIG. 7 is a flow diagram illustrating an example of a testing sequence for the presence of microorganism in a sample.

### DETAILED DESCRIPTION OF THE PREFERRED EMBODIMENT(S)

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

Throughout this application, where publications are referenced, the disclosures of these publications in their entireties are hereby incorporated by reference into this application to more fully describe the state of the art to which this invention pertains.

In an embodiment of the present invention, an apparatus for detecting the presence of a microorganism in a microbial sample is provided. Characteristically, the microorganism produces or consumes a metabolic compound. Advantageously, the present embodiment monitors the concentration(s) of such metabolic compounds. Examples of such metabolic compounds include oxygen, carbon dioxide, alcohols (e.g., ethanol), methane, hydrogen disulfide, and combinations thereof. The microbial sample analyzed by the apparatus of the present embodiment includes the microorganism and a growth medium.

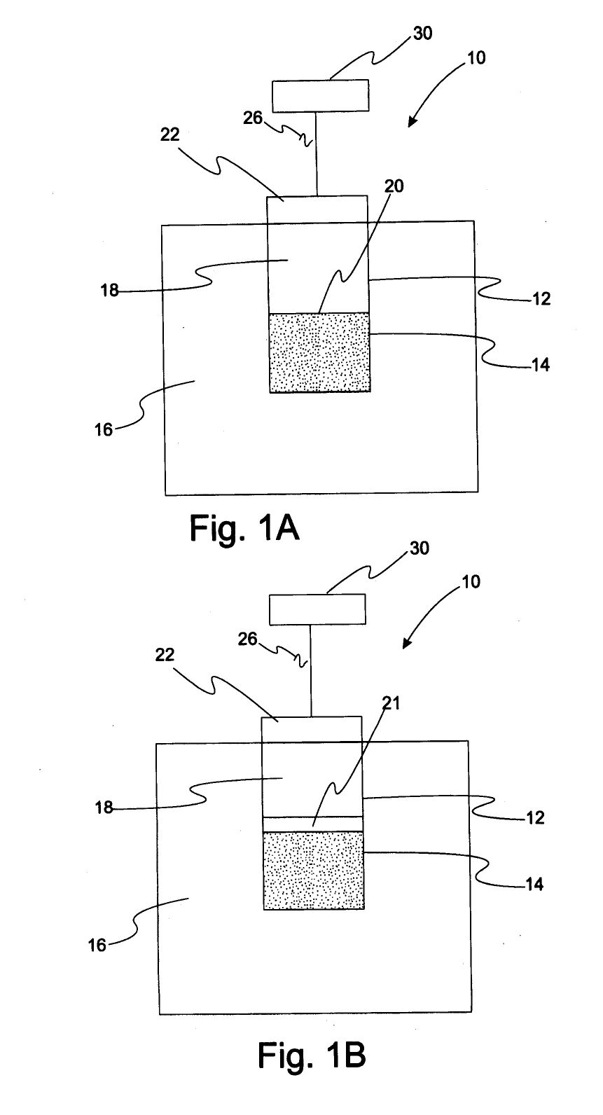

With reference to FIGS. 1A and 1B, schematic illustrations of the microbe-detecting apparatus of the present invention are provided. Microbe-detecting apparatus 10 includes enclosable chamber 12 for holding portion 14 of a microbial sample. In the variations depicted in FIGS. 1A and 1B, at least a portion of enclosable chamber 12 is defined within substrate 16. Examples of suitable substrates include, but are not limited to, glass, quartz, crystalline silicon, thermoplastic polymers, polymer composites, polymer coated metals, and the like. Enclosable chamber 12 holds portion 14 of the microbial sample in a manner that allows porous region 18 to be formed therein, thereby defining interface 20 between porous region 18 and portion 14. In one refinement, porous region 18 is a porous layer. In another refinement, porous region 18 is a gaseous region. In still another refinement, interface 20 is replaced with semipermeable membrane 21 as depicted in FIG. 1B. Ideally, enclosable chamber 12 is substantially impermeable to the metabolic compound. For example, when the metabolic compound is molecular oxygen, enclosable chamber 12 is substantially oxygen impermeable and the coated electrode that contains metabolic compound monitor 22 also contains interface 20 and porous region 18. Microbe-detecting apparatus 10 includes metabolic compound monitor 22 in communication with porous region 18. Metabolic compound monitor 22 provides signal 26 which is functionally dependent on metabolic compound concentration (i.e., the concentration of the metabolic compound(s)) in porous region 18. Signal 26 is receiving by data processing system 30 for analysis.

Signal 26 allows identification of a metabolic compound rich state (i.e., a state with a relatively high concentration of the metabolic compound) and a metabolic compound depleted state (i.e., a state with a relatively low concentration of the metabolic compound) such that at some point during a predetermined period of time a transition between the metabolic compound rich state and the metabolic compound depleted state occurs. In one variation, the metabolic compound is a metabolic product so that the relevant transition is from a depleted state to a rich state. In a refinement of this variation, the metobolic compound is a primary or secondary metabolite. Examples of primary metabolites include fermentation products, nitrite, sulfide, carbon dioxide, and the like. Examples of secondary metabolites include siderophores, quinolones, bacteriocins, colicins, pigments, exotoxins, and the like. In another variation of the present embodiment, the metabolic compound is consumed by the microbe (e.g., a food source, oxygen) so that the relevant transition is from a rich state to a depleted state. In a refinement of this variation, the metabolic compound is a nutrient. Examples of such nutrients include, but are not limited to, carbon and energy sources, minerals and micronutrients, nitrogen sources, and sulfur sources. Specific examples of carbon and energy sources include glucose and other hexoses, glycerol, pyruvate, succinate, fatty acids, amino acids, peptides and the like. Specific examples of minerals and micronutrients include Mg, Ca, Fe, Cu, Zn, vitamins, oleic acid, and the like. Specific examples of nitrogen compounds ammonium, nitrate, nitrite, amino acids, urea, dinitrogen, and the like. Specific examples of sulfur sources include sulfur-containing amino acids, sulfate, sulfite, sulfide, sulfur, and the like. This latter variation is particularly useful for detecting the presence of aerobic microbes which of course consume oxygen. When the microbe is aerobic, the transition from an oxygen rich to oxygen depleted state is advantageously monitored. In still another variation of the present invention, the metabolic compound is a cell constituents. Such cell constituents include intracellular, periplasmic, and extracellular constituents. Examples of intracellular constituents include ATP, DNA, RNA, glucose-6-phosphate dehydrogenase, DNA polymerase, poly-B-hydroxybutyrate, and the like. Examples of periplasmic constituents include acid phosphatase, cyclic phosphodiesterase, ribonuclease I, phosphoglucose isomerase, and the like. Examples of extracellular constituents include phospholipase A, lipopolysaccharide, capsule, and the like.

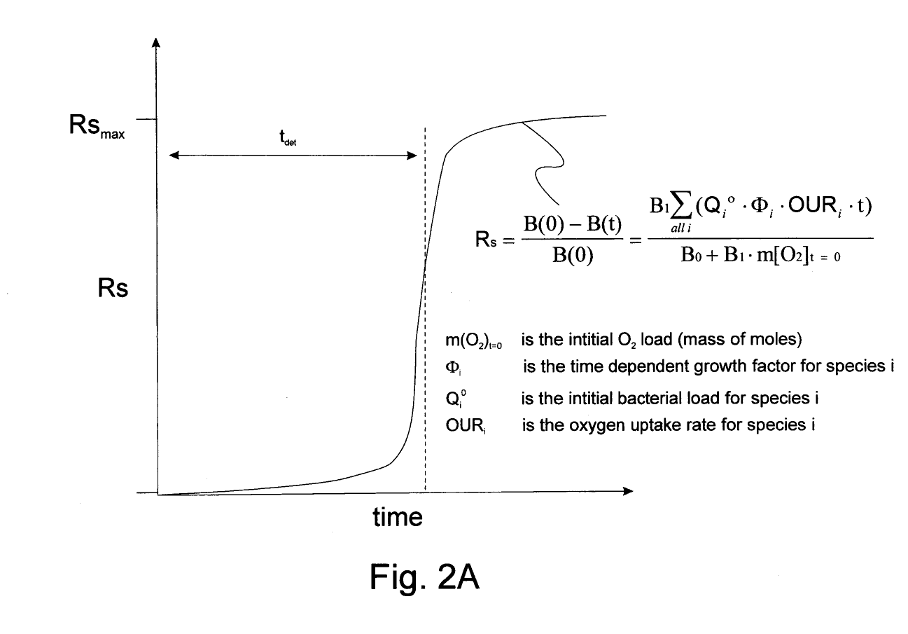

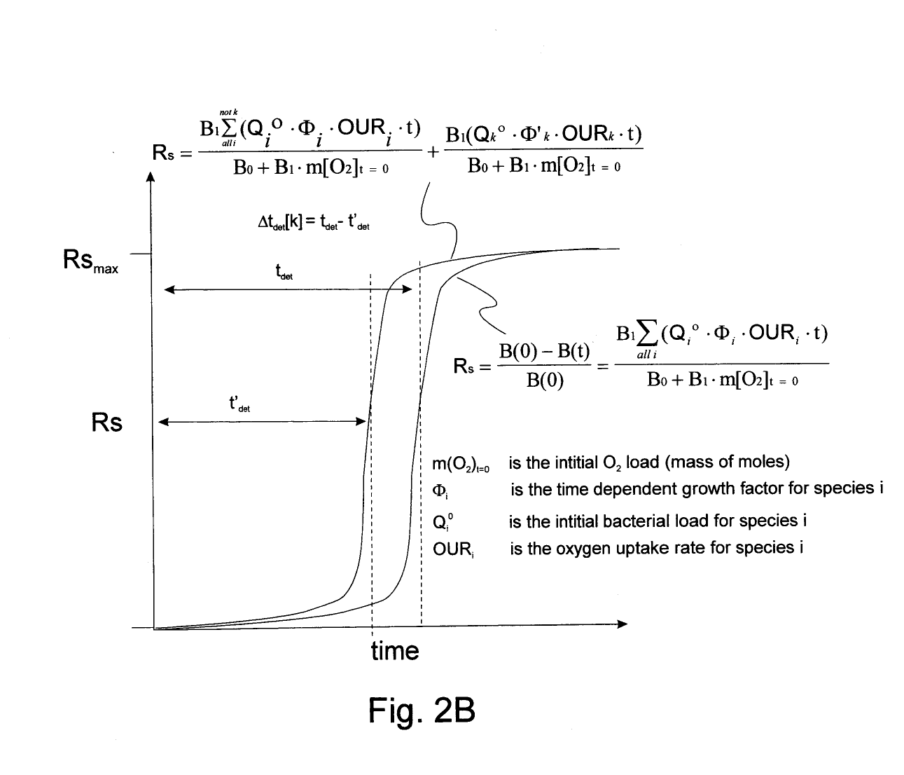

With reference to FIG. 2A, a plot of a useful signal value versus time response is illustrated. In this idealized plot, the signal Rs increases from a low value to a high value. This transition is characterized by a time-to-detection (“t_{d}”). In a variation, the signal Rs given by the following equation (the time-to-detection is the time associated with the inflection point):

$${R}_{s} = \frac{B ( 0 ) - B ( t )}{B ( 0 )} = \frac{{B}_{1} {\sum }_{all i} ( {Q}_{i}^{0} \cdot {Φ}_{i} \cdot {OUR}_{i} \cdot t )}{{B}_{0} + {B}_{1} \cdot {m [ {O}_{2} ]}_{t = 0}}$$

wherein:
m[O₂]ₜ₌₀ is the initial load (mass or moles);
m[O₂](t) is the is the time dependent O₂ load (mass or moles)
Φᵢ is the time dependent growth factor for species i;
Qᵢ⁰ is the initial bacterial load for species i;
OURᵢ is the oxygen uptake rate for species i;
B(t) is a raw unprocessed signal;
B₀, B₁ are instrument constants.

In detecting or quantifying target organisms, it is the time-to-detection that is the most useful quantity and not the absolute oxygen concentration or respiration rate. Therefore, two optimum regimes of testing/cell growth exist:
1. t_{det}<t_{gen}(generation time): Φ=1
2. t_{det}>t_{gen}

In the first regime (Qᵢ⁰ Φᵢ) can be treated as a constant. In the second regime, target organism lysis can occur and differential detection is possible.

With reference to FIG. 2B, plots of useful signal values versus time response is illustrated. In this variation, the signal Rs is calculated as above and the signal R's is calculated as follows:

$${R}_{s} = \frac{{B}_{1} {\sum }_{all i}^{not k} ( {Q}_{i}^{0} \cdot {Φ}_{i} \cdot {OUR}_{i} \cdot t )}{{B}_{0} + {B}_{1} \cdot {m [ {O}_{2} ]}_{t = 0}} + \frac{{B}_{1} ( {Q}_{k}^{0} \cdot {Φ}_{k}^{′} \cdot {OUR}_{k} \cdot t )}{{B}_{0} + {B}_{1} \cdot {m [ {O}_{2} ]}_{t = 0}}$$

where m(O₂)ₜ, m[O₂](t), Qᵢ⁰, B(t), B₀, B₁, OURᵢ are the same as set forth above. Φ′ₖ is the altered time dependent growth factor for species k. Such alteration is caused by impacting the growth of a particular organism more than others in a sample (e.g., bacteriophage, antibiotic, other chemicals, etc.) In this figure, the time to detection differences become the relevant factor.

In a variation of the present embodiment, enclosable chamber 12 is configured to hold portion 14 and porous region 18 in a geometrical relationship and also using the transport properties of interface 20 such that the metabolic compound concentration (e.g., oxygen concentration) is within 10 percent of its equilibrium value within 10 minutes of the enclosable chamber being charged with the microbial sample.

Metabolic compound monitor 22 may utilize any number of methods for measuring the presence of the metabolic compounds in porous region 18. Such methods include, but are not limited to, spectroscopic techniques, electrochemical reaction measurements, impedance measurements, potentiometric measurements, amperometric measurements other electrical measurement techniques, and combinations. When the metabolic compound is oxygen, metabolic compound monitor 22 is an oxygen monitor. In one refinement, the oxygen monitor is operable to measure oxygen-quenching of luminescence emitted by an oxygen-sensing compound.

In a variation of the present embodiment, the microbial sample further comprises a microbial growth-altering material that is specific to a target microorganism. The microbial growth-altering material either enhances or retards the growth of the target microorganism. In one refinement, the microbial growth-altering material is a bacteriophage. Examples of useful bacteriophages include, but are not limited to, DS6A, LG, Gamma phage, FaH, R, {phi}A1122, P 3d, KH1, KH4, and KH5, 212/Hv, BPP-1, A118, A1, A6, phiBB-1, CPL-1, VI 1s5, 34add, VI 1s34add, VI, VI 1s16o, and XIV, 209P.

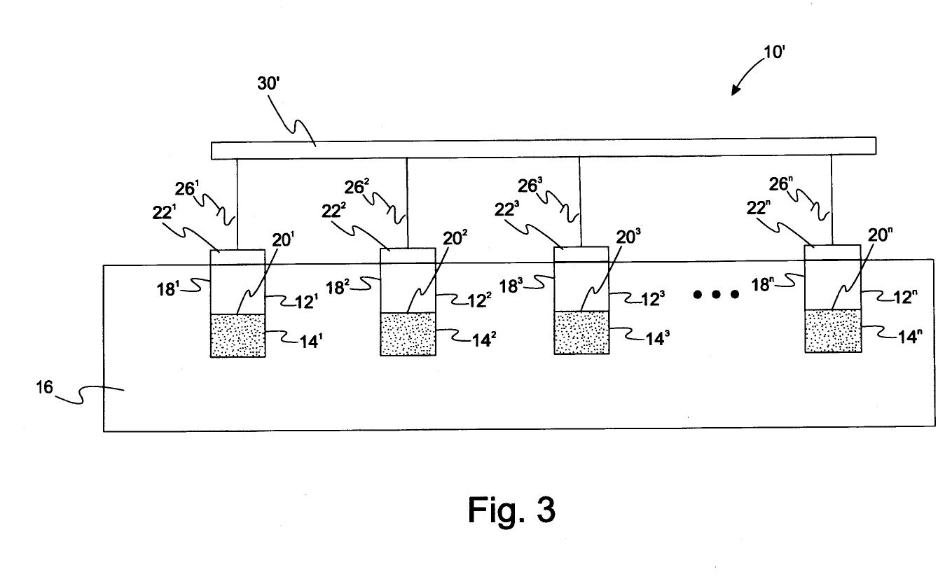

In another embodiment of the present invention, a microbe-detecting apparatus having a plurality of enclosable chambers is provided. With reference to FIG. 3, a schematic illustration of this apparatus is provided. In this embodiment, microbe-detecting apparatus 10′ includes a plurality of enclosable chambers 12″ which are of the general construction set forth above for FIGS. 1A and 1B. Each of the enclosable chambers is at least partially formed within substrate 16. Enclosable chambers 12ⁿ hold portions 14″ of microbial sample(s) in a manner that allows porous regions 18″ to be formed therein thereby defining interface 20ⁿ between porous regions 18ⁿ and portion 14ⁿ. Microbe-detecting apparatus 10′ includes metabolic compound monitors 22ⁿ in communication with porous region 18ⁿ. Metabolic compound monitor 22ⁿ provides signals 26ⁿ which are each functionally dependent on metabolic compound concentration (i.e., the concentration of the metabolic compound(s)) in each respective porous region 18ⁿ through interface 20ⁿ. As set forth above, each of signals 26ⁿ allows identification of a metabolic compound rich state (i.e., a state with a relatively high concentration of the metabolic compound) and a metabolic compound depleted state (i.e., a state with a relatively low concentration of the metabolic compound) such that at some point during a predetermined period of time a transition between the metabolic compound rich state and the metabolic compound depleted state occurs. Signals 26ⁿ are receiving by data processing system 30′ for analysis.

In a variation of the present embodiment, one or more of portions 14ⁿ include an aerobic microorganism. In such variations, the corresponding metabolic compound monitors 22ⁿ are oxygen monitors.

In a particularly useful variation of the present invention, portions 14ⁿ of microbial sample(s) comprises differing compositions. For example, microbe-detecting apparatus 10′ includes a first enclosable chamber 12¹ holds first portion 14¹ of a microbial sample in a manner that allows porous region 18¹ to be formed therein thereby defining interface 20¹ between first porous region 18¹ and the first portion 14¹. Microbe-detecting apparatus 10′ includes first metabolic compound monitors 22¹ in communication with porous region 18¹. Metabolic compound monitor 22¹ first signal 26¹ which is functionally dependent on metabolic compound concentration (i.e., the concentration of the metabolic compound(s)) in first porous region 18¹. Signal 26¹ allows identification of a metabolic compound rich state (i.e., a state with a relatively high concentration of the metabolic compound) and a metabolic compound depleted state (i.e., a state with a relatively low concentration of the metabolic compound) occurring in first porous region 18¹ such that at some point during a predetermined period of time a transition between the metabolic compound rich state and the metabolic compound depleted state occurs. Signals 26¹ are receiving by data processing system 30′ for analysis. In this variation, microbe-detecting apparatus 10′ further includes second enclosable chamber 12² for holding second portion 14² of a microbial sample. Second enclosable chamber 12² holds second portion 14² in a manner that allows second porous region 18² to be formed therein thereby defining interface 20² between second porous region 18² and the second portion 14². Microbe-detecting apparatus 10′ includes second metabolic compound monitor 22¹ in communication with the second porous region 18². Second metabolic compound monitor 22² providing second signal 26² functionally dependent on the metabolic compound concentration of second porous region 18². Signal 26² allows identification of a metabolic compound rich state (i.e., a state with a relatively high concentration of the metabolic compound) and a metabolic compound depleted state (i.e., a state with a relatively low concentration of the metabolic compound) occurring in second porous region 18² such that at some point during a predetermined period of time a transition between the metabolic compound rich state and the metabolic compound depleted state occurs. In this variation, portions 14ⁿ contain samples having different compositions. For instance portion 14¹ include a microbe, a growth medium, and a microbial growth-altering material while portion 14² includes substantially the same composition minus the microbial growth-altering material. Optionally, microbe-detection apparatus 10′ further includes one or more additional enclosable chambers for holding one or more additional portions of the microbial sample and one or more additional metabolic compound monitors in communication with each of the gaseous region as set forth above.

In another variation of the microbe-detecting apparatus of FIG. 3, a portion of the plurality of enclosable chambers 12ⁿ have differing sample volumes. In one refinement of this variation, microbe-detecting apparatus 10′ is operable to determine a usable volume for detecting the presence of the microorganism.

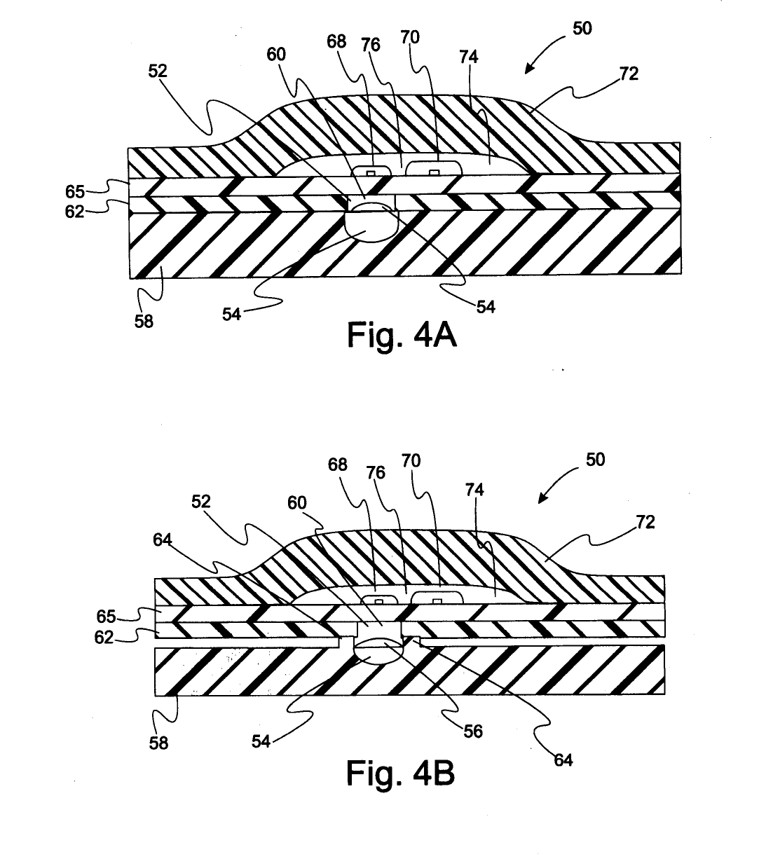

With reference to FIGS. 4A and 4B, another embodiment of an apparatus for detecting the presence of a microorganism in a microbial sample utilizing an electrochemical cell is provided. FIGS. 4A and 4B are cross-sectional views of variations of the present embodiment. Microbe-detecting apparatus 50 includes enclosable chamber 52 for holding growth medium 54 and portion 56 of the microbial sample. In the variation depicted in FIG. 4A, enclosable chamber 52 resides in substrate 58. Examples of suitable substrates include, but are not limited to, glass, quartz, crystalline silicon, thermoplastic polymers, polymer composites, polymer coated metals, and the like. Enclosable chamber 52 holds the portion 54 of microbial sample in a manner that allows gaseous region 60 to be formed therein thereby defining an interface between gaseous region 60 and portion 54 as set forth above. In a refinement of the present embodiment, enclosable chamber 52 is partially defined by spacer 62. In a further refinement of the present embodiment, spacer 62 is substantially oxygen impermeable. FIG. 4B illustrates a variation in which protrusion 64 from substrate 58 also define a portion of enclosable chamber 50. Microbe-detecting apparatus 50 also includes substrate 65 which is disposed over spacer 62. In a further refinement, substrate 65 is oxygen impermeable. The present embodiment also includes a metabolic compound monitor as set forth above. In this embodiment, the metabolic compound monitor is an oxygen monitor. In a particularly useful, refinement, the oxygen monitor utilizes an electrochemical cell. FIGS. 4A and 4B include working electrodes 68 and counter electrodes 70 utilized in such an electrochemical cell. Working electrodes 68 and counter electrodes 70 are disposed over portions of substrate 65, typically adjacent to enclosable chamber 52 so that the metabolite being monitored may easily travel to working electrode 68. Top sheet 72 is disposed over at least a portion of substrate 65 and electrodes 68, 70 in a manner to define space 74. Electrolyte 76 is positioned within space 74. In a variation, electrolyte 76 is a gel electrolyte.

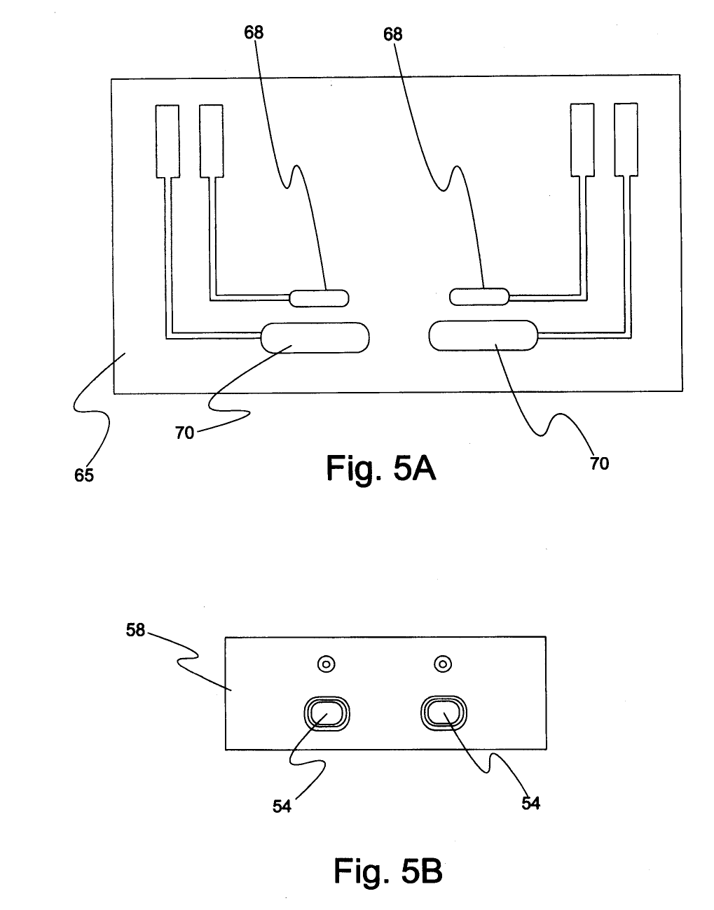

With reference to FIG. 5A, a top view of a substrate with electrodes disposed thereon is provided. As set forth above, working electrodes 68 and counter electrodes 70 are disposed over portions of substrate 65. FIG. 5A depicts a configuration with two sets of electrodes. Electrodes are formed on substrate 65 by any number of methodologies known to those skilled in the art. Examples of such methods include, but are not limited to, screen printing, laser deposition, painting, vacuum deposition, sputtering, chemical vapor deposition and the like.

With reference to FIG. 5B, a top view of a substrate having an enclosable chamber defined therein is provided. As set forth above, enclosable chamber 52 holds growth medium 54 and portion 56 of the microbial sample. FIG. 5B depicts an arrangement with two enclosable chambers. This configuration is designed to line up with the two sets of electrodes depicted in FIG. 5A. Extension of the designs of FIGS. 5A and 5B to situations, additional chambers and electrodes sets are readily appreciated.

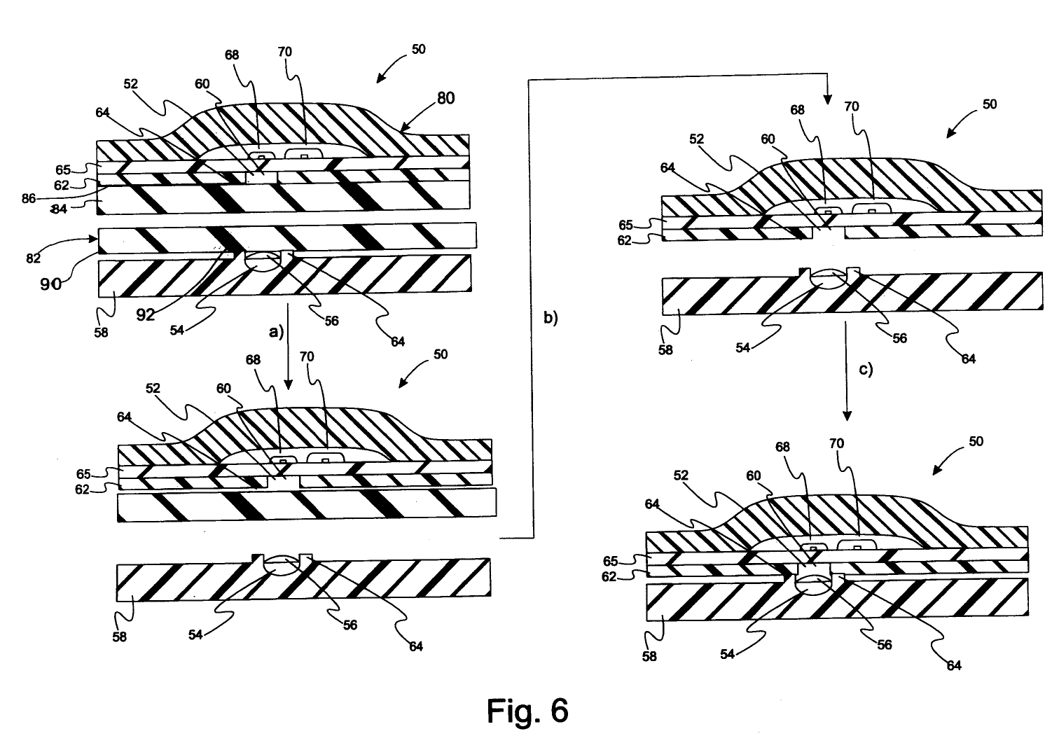

With reference to FIG. 6, a series of cross-sectional views illustrating an embodiment for forming the construction of the enclosable microbe-detecting apparatus set forth above is provided. FIG. 6 illustrates the fabrication for the apparatus of FIG. 4B. This methodology is readily extended to the other embodiments and variations described above. Microbe-detecting apparatus 50 is formed from electrode containing section 80 and growth medium container section 82. Electrode-containing section 80 includes spacer 62, substrate 65, working electrodes 68 and counter electrodes 70, top sheet 72, and electrolyte 76 as set forth above. Protective sheet 84 is disposed over side 86 of electrode-containing section 80. Protective sheet 84 is peeled away just prior to forming microbe-detecting apparatus 50. Growth medium container section 82 includes substrate 58 and growth medium 54. In a similar fashion, protective sheet 90 is disposed over side 92 of growth medium container section 82. Again, protective sheet 90 is peeled away just prior to forming microbe-detecting apparatus 50.

Still referring to FIG. 6, protective sheet 90 is peeled away from growth medium container section 82 in step a) to allow introduction of portion 56 of the microbial sample. In step b), protective sheet 84 is peeled from electrode-containing section 80. Microbe-detecting apparatus 50 is formed in step c) when electrode-containing section 80 is contacted with growth medium container section 82.

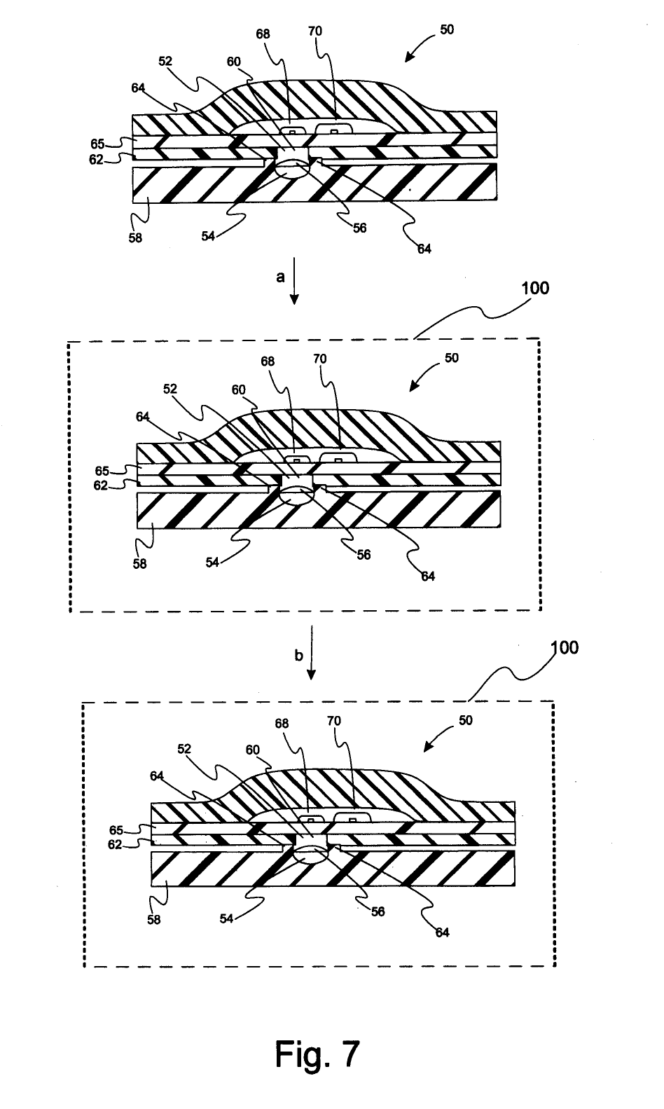

With reference to FIG. 7, a flow diagram illustrating an example of a testing sequence for the presence of microorganism in a sample is provided. Microbe-detecting apparatus 50 is first formed. Apparatus is then inserted into instrumentation-control module 100 in step a). The presence of the metabolite is measured in step b) while the sample is incubated.

In another embodiment of the present invention, a method of detecting a microbe using the apparatuses set forth above is provided. The method of this embodiment comprises charging an enclosable chamber with a sample matrix. The sample matrix includes the microbe and a growth medium. Typically, the sample include a plethora of microbes one of which is the microbe of interest. The enclosable chamber holds the sample matrix in a manner that allows a gaseous region to be formed therein thereby defining an interface between the gaseous region and the sample matrix. A first signal functionally dependent on a metabolic compound concentration of the gaseous region is measured for a predetermined period of time. The signal allows identification of a metabolic compound rich state and a metabolic compound depleted state such that at some point during the predetermined period of time a transition from the metabolic compound rich state to the metabolic compound depleted state occurs (transition time). In a variation, the method further comprises measuring a second signal functionally dependent on the metabolic compound concentration of the gaseous region for a predetermined period of time. A difference between the first and second signals is then determined (a transition time difference). In a refinement, the metabolic compound is oxygen and the microbe is an aerobic microbe.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

1.-23. (canceled)

24. An apparatus for detecting the presence of a microorganism in a microbial sample, the microorganism producing or consuming a metabolic compound, the microbial sample including the microorganism and a growth medium, the apparatus comprising:
an first enclosable chamber for holding a first portion of the microbial sample, wherein the first enclosable chamber holds the first portion of the microbial sample in a manner that allows a first gaseous region to be formed therein thereby defining an interface between the first gaseous region and the first portion; and
a first metabolic compound electrochemical monitor in communication with the gaseous region, the first metabolic compound electrochemical monitor providing a signal functionally dependent on metabolic compound concentration in the first gaseous region wherein the signal allows identification of a metabolic compound rich state and metabolic compound depleted state such that at some point during a predetermined period of time a transition between the metabolic compound rich state and the metabolic compound depleted state occurs.

25. The apparatus of claim 24 wherein the metabolic compound comprises a component selected from the group consisting of oxygen, carbon dioxide, alcohols, methane, hydrogen disulfide, and combinations thereof.

26. The apparatus of claim 24 wherein the microbial sample further comprises a microbial growth-altering material that is specific to a target microorganism, the microbial growth-altering material either enhancing or retarding the growth of the target microorganism.

27. The apparatus of claim 26 wherein the microbial growth-altering material is a bacteriophage.

28. The apparatus of claim 26 further comprising:
a second enclosable chamber for holding a second portion of the microbial sample, the second portion not including the microbial growth-altering material, wherein the second enclosable chamber holds the second portion in a manner that allows a second gaseous region to be formed therein thereby defining an interface between the second gaseous region and the second portion; and
a second metabolic compound electrochemical monitor in communication with the second gaseous region, the second oxygen electrochemical monitor providing a signal functionally dependent on the oxygen concentration of the second gaseous region wherein the signal allows identification of an oxygen rich state and an oxygen depleted state such that at some point during a predetermined period of time a transition from the oxygen rich state to the oxygen depleted state occurs.

29. The apparatus of claim 24 further comprising:
one or more additional enclosable chambers for holding one or more additional microbial sample portions, each portion being held in a manner that allows a corresponding gaseous region to be formed therein thereby defining an interface between each portion and the corresponding gaseous region.

30. The apparatus of claim 29 further comprising:
one or more metabolic compound electrochemical monitors in communication, each corresponding gaseous region having an associated oxygen electrochemical monitor from the one or more additional oxygen electrochemical monitors, each associated oxygen electrochemical monitor providing an associated signal functionally dependent on the oxygen concentration of the corresponding gaseous regions wherein the associated signal allows identification of an oxygen rich state and an oxygen depleted state such that at some point during a predetermined period of time a transition from the oxygen rich state to the oxygen depleted state occurs.

31. The apparatus of claim 24 wherein the enclosable chamber is configured to hold the first portion and the gaseous region in a geometrical relationship such that the oxygen concentration is within 10 percent of its equilibrium value within 10 minutes of the enclosable chamber being charged with the sample matrix and closed.

32. An apparatus for detecting the presence of an aerobic microorganism in a microbial sample, the microbial sample including the aerobic microorganism and a growth medium, the apparatus comprising:
a first enclosable chamber for holding a first portion of the microbial sample, wherein the first enclosable chamber holds the first portion of the microbial sample in a manner that allows a first gaseous region to be formed therein thereby defining an interface between the first gaseous region and the first portion; and
a first oxygen electrochemical monitor in communication with the gaseous region, the first oxygen electrochemical monitor providing a signal functionally dependent on the oxygen concentration of the first gaseous region wherein the signal allows identification of an oxygen rich state and an oxygen depleted state such that at some point during a predetermined period of time a transition from the oxygen rich state to the oxygen depleted state occurs.

33. The apparatus of claim 32 wherein the microbial sample further comprises a microbial growth-altering material that is specific to a target microorganism, the microbial growth-altering material either enhancing or retarding the growth of the target microorganism.

34. The apparatus of claim 33 wherein the microbial growth-altering material is a bacteriophage.

35. The apparatus of claim 33 further comprising:
a second enclosable chamber for holding a second portion of the microbial sample, the second portion not including the microbial growth-altering material, wherein the second enclosable chamber holds the second portion in a manner that allows a second gaseous region to be formed therein thereby defining an interface between the second gaseous region and the second portion; and
a second oxygen electrochemical monitor in communication with the second gaseous region, the second oxygen electrochemical monitor providing a signal functionally dependent on the oxygen concentration of the second gaseous region wherein the signal allows identification of an oxygen rich state and an oxygen depleted state such that at some point during a predetermined period of time a transition from the oxygen rich state to the oxygen depleted state occurs.

36. The apparatus of claim 32 further comprising:
one or more additional enclosable chambers for holding one or more additional portions of the microbial sample, wherein each chamber of the one or more additional enclosable chambers hold one portion of the one or more additional portions, each portion being held in a manner that allows a corresponding gaseous region to be formed therein thereby defining an interface between each portion and the corresponding gaseous region.

37. The apparatus of claim 36 further comprising:
one or more additional oxygen electrochemical monitors in communication, each corresponding gaseous region having an associated oxygen electrochemical monitor from the one or more additional oxygen electrochemical monitors, each associated oxygen electrochemical monitor providing an associated signal functionally dependent on the oxygen concentration of the corresponding gaseous regions wherein the associated signal allows identification of an oxygen rich state and an oxygen depleted state such that at some point during a predetermined period of time a transition from the oxygen rich state to the oxygen depleted state occurs.

38. The apparatus of claim 32 wherein the gaseous region includes molecular oxygen.

39. The apparatus of claim 32 wherein the enclosable chamber is oxygen impermeable.

40. The apparatus of claim 32 wherein the first enclosable chamber is configured to hold the sample matrix and the gaseous region in a geometrical relationship such that the oxygen concentration is within 10 percent of its equilibrium value within 10 minutes of the enclosable chamber being charged with the sample matrix and closed.

41. An apparatus for detecting the presence of an aerobic microorganism in a microbial sample, the microbial sample including the aerobic microorganism and a growth medium, the apparatus comprising:
a plurality of enclosable chambers, wherein each chamber of the plurality of enclosable chambers holds a corresponding portion of the microbial sample in a manner that allows an associated gaseous region to be formed within each chamber thereby defining in each chamber an interface between the gaseous region and the corresponding portion; and
a plurality of oxygen electrochemical monitors in communication with each gaseous region, the each electrochemical monitor of the plurality of oxygen electrochemical monitors providing a signal functionally dependent on the oxygen concentration of the first gaseous region wherein the signal allows identification of an oxygen rich state and an oxygen depleted state such that at some point during a predetermined period of time a transition from the oxygen rich state to the oxygen depleted state occurs.

42. The apparatus of claim 41 wherein a portion of the plurality of enclosable chambers have differing sample volumes.
